(19) United States
(12) Patent Application Publication
(10) Pub. No.: US 2003/0100940 A1
(43) Pub. Date: May 29, 2003

(54) Implantable intraluminal protector device and method of using same for stabilizing atheromas

(75) Inventor: Ofer Yodfat, Modl'in (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: MindGuard Ltd.

(21) Appl. No.: 10/288,443

(22) Filed: Nov. 6, 2002

Related U.S. Application Data
(60) Provisional application No. 60/332,048, filed on Nov. 23, 2001.

(51) Int. Cl. A61F 2/06

(52) U.S. Cl. 623/1.15

(57) ABSTRACT

An intraluminal device implantable in a body lumen having an atheroma therein in the vicinity of a side-branch vessel includes a mesh-like tube of bio-compatible material formed with liquid-permeable window openings. The mesh-like tube has an expanded condition in which the tube diameter is slightly larger than the diameter of the body lumen in which it is to be implanted, and the tube length is sufficient to cover the atheroma and the side-branch orifice, and to be anchored to the body lumen around the periphery of the atheroma, and a contracted condition wherein it is sufficiently flexible so as to be easily manipulatable through the body lumen to the site of the atheroma. The mesh-like tube, in its expanded condition, has window openings of a size and distribution such as to structurally stabilize the atheroma and to keep embolic material originating from the atheroma in place on the wall of the body lumen, while diverting embolic material of predetermined size present in the blood flowing through the mesh-like tube from the side-branch orifice, without substantially impeding the blood flow, or increasing the thrombogenitic properties, of the blood flowing into the side-branch orifice.

This application claims the benefit of priority from U.S. Provisional Patent Application No. 60/332,048, filed Nov. 23, 2001, the contents of which are incorporated herein by reference.

### FIELD OF THE INVENTION

The present invention relates to implantable intraluminal devices and methods of using such devices in stabilizing atheromas in a body lumen. More particularly, the present invention is related to devices that are placed in the aortic arch to stabilize atheromas in case of aortic arch atheroma, most particularly protruding aortic arch atheroma and/or ascending aortic arch atheroma and/or mobile aortic arch atheroma and/or aortic proximal arch atheroma, and to prevent the emboli originating from atheromas and other proximal sources from entering, via the side-branches of the aortic arch, arteries that carry blood to the brain.

### BACKGROUND OF THE INVENTION

Publications and other reference materials referred to throughout are incorporated herein by reference in their entirety and are numerically referenced in the following text and respectively grouped in the appended Bibliography which immediately precedes the claims.

In recent years, the presence of protruding atheromas in the aortic ascending (proximal) arch has been recognized as a potential cause of cerebral or peripheral embolization in the elderly [1-2]. The significance of the aorta as a source of emboli has become apparent since the advent of transesophageal echocardiography (TEE), and considerable data has been published on the high recurrence rate of cerebral or peripheral emboli in patients with protruding atheroma of the aortic arch identified by the use of TEE.

Better visualization of the aortic arch wall has enabled physicians to classify atheromatous aortic plaque according to position, thickness, degree of intraluminal protrusion, presence of ulceration on its luminal surface, and presence or absence of mobile components [3]. Large atheromatous plaques tend to ulcerate with a subsequent rupture or thrombus formation with a high probability of dislodging emboli. These phenomena can occur either spontaneously, or during an aortic manipulation (e.g., cardiac catheterization or cardiac surgery).

Several case-control studies with autopsy findings have identified large atheromas, of more than 4-5 millimeter thickness, as one of the most powerful independent risk factors for ischemic stroke in patients older than 60 years of age [4, 5].

The presence of aortic atheroma increases sharply with age, from 20% in the fifth decade to 80% over the age of 75 years. The prevalence of atheromas in the aortic arch in patients with an embolic disease is between 21-27%, in contrast to 4-13% in control subjects. In the presence of atheromas, the incidence of a future stroke and peripheral emboli was 33% in a single year [2].

The prevalence of aortic atheromas among the patients who had ischemic stroke is of about the same magnitude as the prevalence of carotid disease (10-13%) and a trial fibrillation (18-30%).

The current treatments of large aortic atheromas include anticoagulant and cholesterol reduction drugs. However, such drugs, in fact, have been noted to prevent only partially the release of thromboembolic particles from these atheromas. Treatment with such drugs is also contraindicated in a large sector of the population at risk. Treatment with drugs along, therefore, cannot provide a satisfactory solution to the problem of atheromatous plaques.

Another treatment approach is aortic endarterectomy as a primary procedure for patients with a previous embolic event. It follows, from observation of a large group of patients [6], that aortic arch endarterectomy greatly increases the risk of intra-operative stroke, and this procedure should not be performed on a routine basis.

Another treatment approach is disclosed in U.S. Pat. No. 6,258,120. The device tends to divert the emboli which come from proximal sources but does not stabilize the aortic arch atheromas itself, since it is not in contact with the entire length of the aortic wall. Methods described for anchoring the device to the aortic wall involve invasive techniques such as installing hooks or sutures at the proximal end of the device, which can cause injury to the vessel wall. In addition, having its distal end being free to move by the emmense pulsative flow forces at the proximal arch, this device is expected to increase the shedding of emboli material from the arch atheroma.

A need therefore exists for a device that prevents the complications arising from the presence of aortic plaques (rupture, thrombi formation and distal embolization) by stabilization of the atheroma and by diverting embolic material away from the the side-branches of the aortic arch which norish the brain.

### OBJECTS AND BRIEF SUMMARY OF THE INVENTION

A broad object of the present invention is to provide an implantable intraluminal protector device that stabilizes atheromas.

Another object of this invention is to provide a protector device capable of filtering the emboli originating from proximal sources, and preventing them from entering the side-branches of the aortic arch that carry blood to the brain, i.e., brachiocephalic trunk, the left common carotid, and the left subclavian, without increasing the thrombegenic properties of the blood passing through the device.

A further object of this invention is to provide a protector device that reduces the possibility of complications arising from the presence of aortic plaques (rupture, thrombi release and distal emobilation) during the course of and following a medical procedure.

A further object of the invention is to provide a device of the foregoing type which can be implanted using minimal invasive techniques.

A still further object is to provide a method of stabilizing atheromas in body lumens in general, and particularly in the aortic arch, and for preventing embolic material from being detached and/or from entering blood vessel side-branches.

According to one aspect of the present invention, there is provided an implantable intraluminal device implantable in a body lumen having an atheroma therein in the vicinity of a side-branch orifice, comprising: a mesh-like tube of bio-compatible material formed with liquid-permeable window openings; the mesh-like tube having an expanded condition in which the tube diameter is slightly larger than the diameter of the body lumen in which it is to be implanted, and the tube length is sufficient to cover the atheroma the side-branch orifice, and to be anchored to the body lumen around the periphery of the atheroma; the mesh-like tube also having a contracted condition wherein it is sufficiently flexible so as to be easily manipulatable through the body lumen to the site of the atheroma; the mesh-like tube, in its expanded condition, having window openings of a size and distribution such as to structurally stabilize the atheroma and to keep embolic material originating from the atheroma in place on the wall of the body lumen, while diverting embolic material of predetermined size present in the blood flowing through the mesh-like tube from the side-branch orifice, without substantially impeding the blood flow, or increasing the thrombogenitic properties, of the blood flowing into the side-branch orifice.

In the described preferred embodiments of the invention, the mesh-like tube also has a porosity index in the expanded condition of the mesh-like tube preferably within the range of 65-95%, more preferably 80-90%. The porosity index (P.E.) is defined by the relation:

$$P \cdot E \cdot = 1 - \frac{S m}{S t}$$

wherein: “Sm” is the actual surface covered by the mesh-like tube, and “St” is the total surface area of the mesh-like tube.

In addition, the window openings in the expanded condition of the mesh-like tube are of a length along one side preferably within the range of 100-1500 μm, more preferably μm, 300-1500, most preferably 400-1000 μm, and have an inscribed diameter preferably of 30-800 μm, more preferably of 30-480 μm, still more preferably 50-320 μm.

According to further features in the preferred embodiments of the invention described below, the mesh-like tube is composed of a braid of filaments. The number of filaments in the braid is preferably within the range of 60-300, more preferably 100-160. Preferably, the filaments have a circular cross-section of a diameter within the range of 20-500 μm, preferably 25-250 μm, more preferably 50-100 μm; however, the filaments may also have a non-circular cross-section, preferably of a circumference within the range of 60-800 μm.

While in the described preferred embodiments, the mesh-like tube is in the form of a braided tube made of a plurality of filaments extending helically in an interlaced manner in opposite directions, it is contemplated that other mesh-like structures could be used, such as woven tubes, knitted tubes or cellular tubes formed, for example, by the removal of material from a non-porous tube to form windows therein. The filaments may be of a suitable bio-compatible material, and may include a drug or other biological coating or cladding. Preferably the mesh-like tube is made of a material selected from the group consisting of 316L stainless steel tantalum, superelastic Nitinol, cobalt base alloy, mixtures of such metals and alloys, bio compatible plastic and complex materials.

According to another aspect of the present invention, there is provided a method for stabilizing an atheroma in a body lumen and preventing embolic material from being detached from atheroma, without substantially impeding the flow of blood through the lumen and a side-branch branching therefrom, the method comprising: implanting an expandable mesh-like tube of a bio-compatible material in the body lumen to cover the atheroma; the mesh-like tube having a contracted state of a first diameter, and an expanded state of a second diameter greater than the first diameter; the mesh-like tube being flexible in its contracted state for manipulation through the body lumen to the implantation site and being suitable to keep the embolic material in place on the wall of the body lumen, to divert embolic material flowing through the tube like structure from the orifice of the side branch, without substantially impeding the blood flow, or increasing the thrombogenitic properties, of the blood flowing into the side-branch orifice. In the described preferred embodiment, the body lumen is the aortic arch.

As will be described more particularly below, intraluminal devices constructed in accordance with the foregoing features show great promise for stabilizing atheromas in a body lumen, particularly in the aortic arch, and preventing embolic material from being detached from the wall of the body lumen, without substantially impeding the flow of blood through the lumen and the side-branches.

Further features and advantages of the invention will be apparent from the description below.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a side view illustrating one form of an implantable intraluminal protector device constructed in accordance with the present invention, the device being shown in its normal, expanded condition;

FIG. 2 is a corresponding view of FIG. 1 illustrating the device in its contracted, stressed condition;

FIG. 3 is a fragmentary view more particularly illustrating the braid pattern in the device of FIGS. 1 and 2;

FIG. 4 schematically illustrates an atheroma in an aortic arch;

FIG. 5 schematically illustrates the intraluminal device implanted in the aortic arch to stabilize the atheroma and to prevent embolic material from entering the side-branches of the aortic arch;

FIG. 6 is a fragmentary view illustrating the manner in which the implanted device stabilizes the atheroma in the aortic arch of FIGS. 3-5;

FIG. 7 schematically illustrates the manner in which the implanted device not only stabilizes the atheroma in the aortic arch, but also prevents embolic material from entering the blood flow into the side-branches without impeding the blood flow through the aortic arch; and

FIG. 8 illustrates another implantable intralumen device constructed in accordance with the present invention for stabilizing an atheroma in a body lumen.

It is to be understood that the foregoing drawings, and the description below, are provided primarily for purposes of facilitating understanding the conceptual aspects of the invention and various possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

The present invention is of an implantable intraluminal devices and methods of using such devices in stabilizing atheromas in a body lumen. The present invention is particularly of devices that are placed in the aortic arch to stabilize atheromas in case of aortic arch atheroma, most particularly protruding aortic arch atheroma and/or ascending aortic arch atheroma and/or mobile aortic arch atheroma and/or aortic proximal arch atheroma, to prevent the emboli originating from atheromas and other proximal sources from entering, via the side-branches of the aortic arch, arteries that carry blood to the brain.

The principles and operation of the devices and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

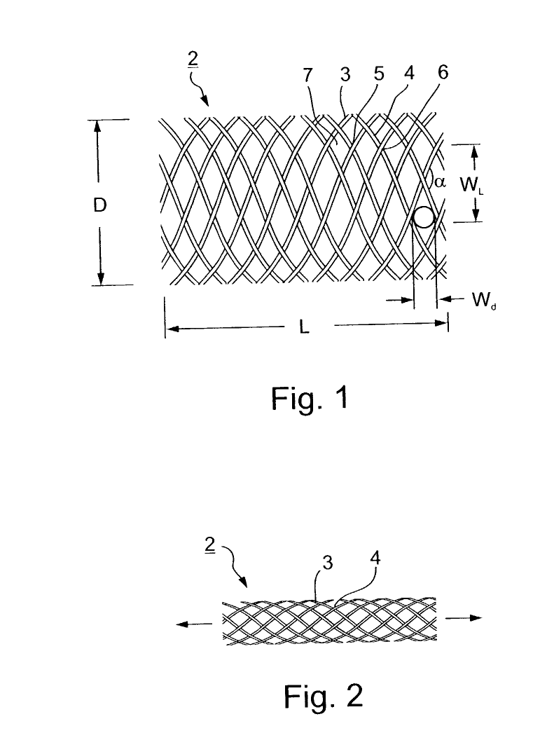

Referring now to the drawings, FIG. 1 illustrates an intraluminal protector device, therein generally designated 2, constructed in accordance with the present invention in its normal or expanded condition which it assumes in a body lumen, particularly the aortic arch, after deployment therein; whereas FIG. 2 illustrates the intraluminal protector device 2 of FIG. 1 in the contracted or stressed condition the device assumes to facilitate its manipulation through the body lumen to the deployment site.

As shown particularly in FIG. 1, the intraluminal protector device 2 is a mesh-like tube made of a plurality of filaments of elastic material, metal or plastic, extending helically in an interlaced manner to define a braided tube. Thus, shown in FIG. 1 is a first group of filaments 3 extending helically in one direction, and a second group of filaments 4 extending helically in the opposite direction, with the two groups of filaments being interwoven such that a filament 3 overlies a filament 4 at some points as shown at 5, and underlies a filament 4 at other points as shown at 6.

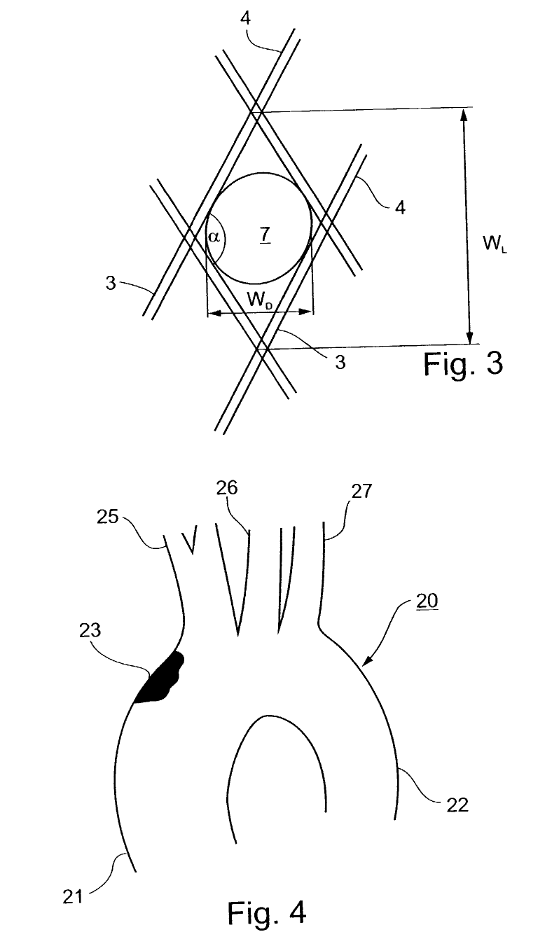

Filaments 3 and 4 thus define a braided tube having a plurality of windows 7. The inscribed diameter and the length of each window are shown in FIG. 3 at W_{d} and W_{L}, respectively, in the normal, expanded condition of the braided tube. These characteristics depend on, among other factors, the number of filaments and the braiding angle “α” at the cross-over points of the two groups of filaments 3, 4.

Such braided-tube intraluminal devices are well-known, for example as described in Wallsten et al., U.S. Pat. No. 5,061,275 and Wallsten, U.S. Pat. No. 4,954,126, the contents of which are incorporated herein by reference. They are generally used as stents for providing support to a wall of a blood vessel, for implanting a graft, e.g., to treat an aneurysm, or for other purposes. As known, the braided tube is normally formed in an expanded condition (FIG. 1) having a diameter slightly larger than the diameter of the blood vessel so that when the device is deployed it becomes firmly embedded in the wall of blood vessel. However, the braided tube is capable of being stressed into a contracted condition, as shown in FIG. 2, wherein the diameter of the braided tube is decreased, and its length increased, to permit manipulation of the braided tube through the blood vessel to the site of implantation.

Further information concerning the construction and deployment of such braided-tube intraluminal devices is available in the above-cited patents, and also in Applicant's International Patent Application PCT/IL01/00624, published Jan. 24, 2002, International Publication No. WO 02/05729, the entire contents of which are incorporated herein by reference.

According to the present invention, the braided tube 2 is constructed for use in stabilizing an atheroma in a body lumen, particularly in the aortic arch, and in preventing embolic material from being detached from the atheroms, without substantially impeding the flow of blood through the lumen and through a side-branch or branches branching therefrom. For this purpose, the braided tube 2 is constructed to have an expanded condition in which the tube diameter is slightly larger than the diameter of the body lumen in which it is to be implanted, and the tube length is sufficient to cover the atheroma and the side-branch or branches orifice or orifices, and to be anchored to the body lumen around the periphery of the atheroma. The construction of the braided tube is such that, in its contracted condition, it is sufficiently flexible so as to be easily manipulatable through the body lumen to the site of the atheroma; and in its expanded condition, it has window openings of a size and distribution such as to structurally stabilize the atheroma and keep any embolic material in place on the wall of the body lumen, while diverting embolic material of predetermined size present in the blood flowing through the mesh-like tube from the side-branch orifice, without substantially impeding the blood flow, or increasing the thrombogenitic properties, of the blood flowing into the side-branch(es) orifice(s). In particular, diversion of embolic material which is of sufficient size to block small blood vessels, such as vessels in the brain, the blocking thereof causes stroke, is envisaged. It will be appreciated that the sources of emboli can be a shedding plaque, thrombi, etc.

In order to ensure proper functioning of the braided tube as such a protector device, and particularly not to increase the thrombogenitic properties of the blood, which among other factors is dependent on the dimensions devices placed in the blood stream, the following preferred dimensional parameters should be observed in the construction of the braided tube:

The porosity index of the braided tube protector, in its expanded condition, is preferably 65-95%, more preferably 80-90%.

The long-dimension length of side W_{L} of the window 7, after expansion, is preferably between 100-1500 μm, more preferably 300-1500 μm, most preferably 400-1000 μm.

The diameter “D” and length “L” in the expanded state of the tube, as shown in FIG. 1a, will vary according to the location and anatomical dimensions of the specific patient; “L” is typically between 10 mm and 40 mm.

The number of filaments to be braided is also a function of the dimensions of the window that it is desired to achieve; preferably this number is in the range of 60-300 filaments more preferably 100-160.

The dimensions of the filaments are as follows: when circular in cross-section diameter “d” is preferably 20-500 μm, preferably 25-250 μm, more preferably 50-150 μm; with cross-sections other than round, the circumference of the filaments is 60-800 μm.

The filaments can be made of any suitable material which is bio-compatible and which can be worked into a braid. Bio-compatible means any material that can be safely introduced and implanted in human or animal bodies for indefinite periods of time without causing any significant physiological damage.

According to a preferred embodiment of the invention, the filament is made of a material selected from among the 316L stainless steel tantalum, superelastic Nitinol, cobalt base alloy, mixtures of such metals and alloys, bio compatible plastic and complex materials. The filament can of course be coated with bio-compatible coatings.

The braided tube 2 has an essentially cylindrical shape with large sections of its body generally serving as an anchoring portion. An anchoring portion is a portion of the device that firmly contacts the walls of the lumen. Such contact causes the wall cell growth into the net of the device and strongly anchors it to the lumen, thus preventing its migration. The physiological processes leading to such anchoring are well known in the art, and will therefore not be discussed herein in detail, for the sake of brevity.

A well known characteristic property of expandable devices of this type is that the device elongates as it is compressed from an expanded (nominal) to a contracted (luminal) state for insertion into the body lumen, i.e., the length in the contracted state is longer than that in the expanded state by an amount that typically varies by 130-500%. The device in the expanded state will create a radial force which will allow it to bond smoothly and flexibly to the vessel wall while covering the atheromas and orifice(s). In the presently preferred embodiment, the aortic arch artery diameter will dictate the size of the protector device. Preferably, it would be dimensioned to exert an average radial force above 150 pascals, e.g., 150-750 pascals on the wall of the aortic arch, or other body lumen in which it is to be implanted.

The illustrated protector device is designed to be introduced subcutenously and moved through the vascular system to the location where it is to be deployed. Introduction of the device into the vascular system and guiding it to the desired location are accomplished by using standard equipment and techniques. These techniques including solutions to the problem of radio-opacity of the device, as well as delivery systems based on these techniques, are extensively discussed in the above mentioned International Patent Application PCT/IL01/00624, incorporated herein by reference.

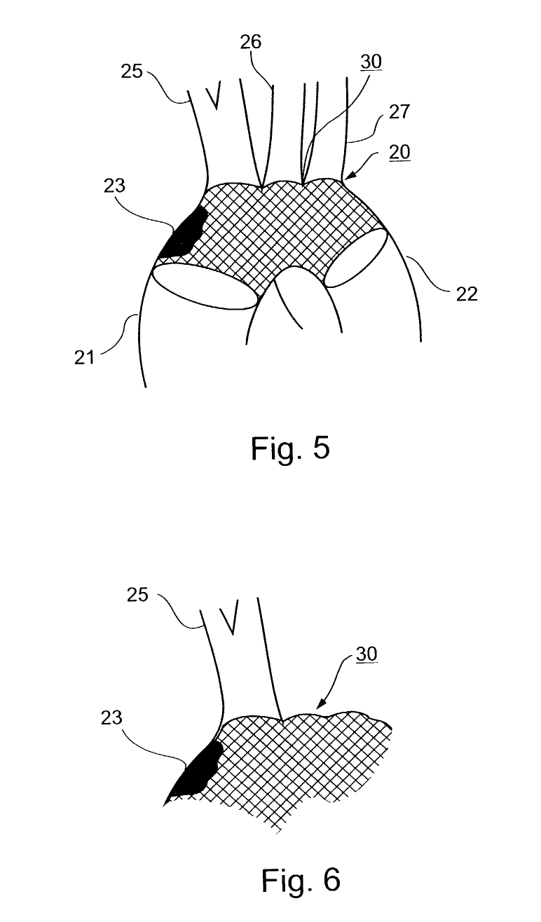

FIGS. 4 and 5 show schematically: the aortic arch 20 including the ascending aorta 21 and the descending aorta 22, a typical atheroma 23 in the ascending aorta 21, and the adjacent side-branch blood vessels, including the brachiocephalic trunk 25, the left common carotid 26, and the left subclavian 27.

FIG. 5 schematically illustrates the protector device 30 implanted in a typical position in the aortic arch 20. In this implanted position, it smoothly and flexibly covers the athorema 23, the walls of the aortic arch 20, and the orifices of the side-branch arteries 25, 26, 27. When so implanted, the protector device 30 is effective to stabilize the atheroma 23 and to keep embolic material originating from the atheroma in place on the wall of the aortic arch 20, while diverting embolic material of predetermined size present in the blood flowing through the device from the side-branches 25, 26 and 27, without substantially impeding the blood flow, or increasing the thrombogenitic properties, of the blood flowing into the side-branches 25, 26 and 27, through their respective orifices.

While the self-expandable device 30 is well suited for implantation, the irregular shape of the aortic arch, and particularly the necessity of tightly enclosing the atheroma between the wall of the aortic arch and the braid of the device, will in some cases dictate the use of an auxiliary device to assist in the final expansion of the device. The auxiliary devices can be part of any delivery system which is capable of supplying the radial forces required to firmly implant the protector. When using such auxiliary devices in the aortic arch artery, it is essential to use devices that do not block the blood flow completely during the expansion, since complete blocking of the blood flow at the aortic arch level will block the blood flow to all the body including the brain, and the results of such an action can be fatal.

Technical solutions to the problem of expanding the device without blocking the artery are extensively discussed in a co-pending U.S. Provisional Patent Application Serial No. 60/322,057 filed Nov. 23, 2001 the description of which is incorporated herein by reference. That application described existing methods and devices as well as a new device, that can be used to aid in deploying either a self-expandable, or non-self-expandable, intraluminal devices in critical areas of the vascular system wherever any stoppage in the flow of blood during the deployment of the interluminal device is not possible or not recommended.

FIG. 6 shows how the fully deployed protector device 30 surrounds and covers the atheroma 23. Since the protector device 30 is designed to exert an average radial force of 150-750 pascal, and since a neointimal cell growth takes place through the mesh-like tube of the device within a very short time, the atheroma 23 is thus stabilized, and subsequent migration of emboli from the atheroma is blocked. With the atheroma stabilized, it can be seen from FIG. 6 that, as a consequence of the braided flexible nature of the implanted protector device, medical procedures in the heart or in the aorta can now be more safely carried out by passing catheters and other medical devices through the length, or through the braided walls, of the implanted protector device.

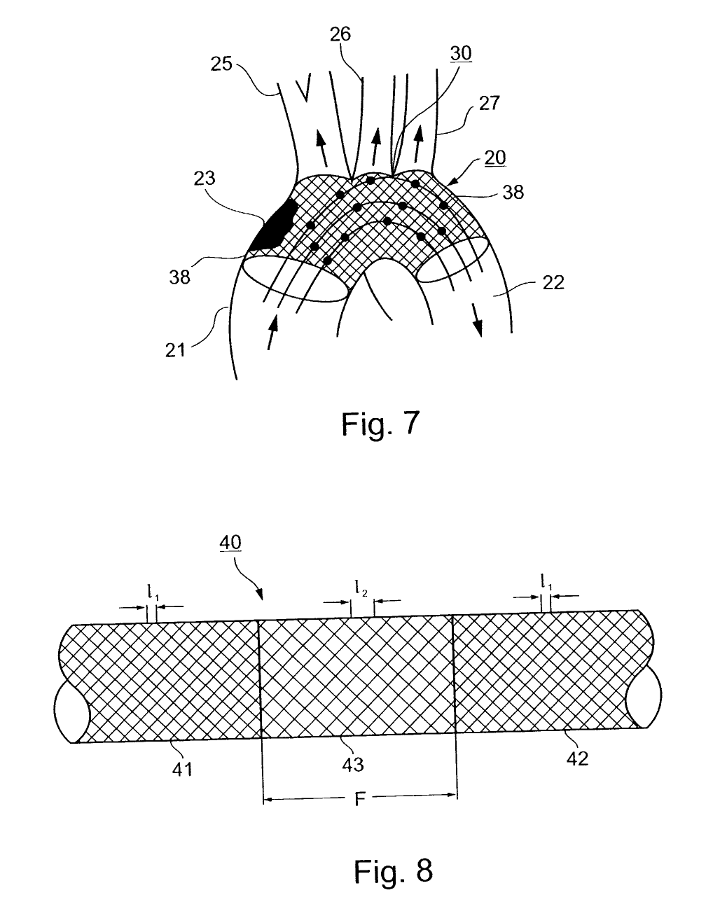

The flexibility of the implanted protector device resulting from its braided nature allows it to adjust itself to the changing shape of the aortic arch spatially, as well as dynamically, i.e., to the changes resulting from the pulsatile flow.

Where the implanted protector device covers one or more of the side-branches as shown in FIGS. 4-6, the wall of the device will act as a diverter that will divert any embolic material of a proximal origin from entering the side-branches. This is illustrated in FIG. 7 wherein the lines and dots 38 schematically represent emboli from proximal locations and flow lines through the aorta, and the arrows show the direction of blood flow in the arteries. The diversion is effected in such a way so as to divert embolic material of proximal origine of predetermined size present in the blood without substantially impeding the blood flow, or increasing the thrombogenitic properties, of the blood flowing into the side-branch orifice(s).

The flexibility of the implanted protector device 30 allows surgical procedures to be carried out in either the main or side-branches since medical devices can be passed through either the center, or even through the wall of the implanted protector device. Thus, the protector device, when implanted as described above, presents no obstacles to an approach of a catheter to the carotid through a percutaneous groin access, if such an approach is necessary.

In an aortic arch, the diameter of the lumen does not vary significantly over the length of the protector device. However, it may be desired to implant the protector device in an artery at a location having different diameters at the two extremities of the protector device. As will be appreciated by one skilled in the art, if a constant diameter device is inserted into such a variable-diameter lumen, this may result in a defective anchoring of the device at the larger diameter lumen, and in a possible risk of migration of the device. Methods of producing braided devices with variable diameters along their longitudinal axis are described in detail in the above-cited International Patent Application PCT/IL01/00624, and incorporated herein by reference, and may be used to overcome the above problem of secure anchoring in variable-diameter lumens.

The novel implantable protector device of the present invention can be constructed in a way very similar to conventional braided stents. Typically, the braid is produced by winding one or more filaments over and under one or more other filaments, or the same filaments, in an interlaced manner as it is wound about a cylinder, cone or contoured mandrel, at constant or variable orientation angles, porosity indices and radii, as described in the above-cited International Patent Application PCT/IL01/00624, incorporated herein by reference.

FIG. 8 illustrates an implantable protector device in accordance with the present invention but based on a construction described in the above-cited International Patent Application, which construction may be used to stabilize the atheroma, while at the same time maximizing the flow of ambolic material free blood into the side-branches, without substantially impeding the blood flow, or increasing the thrombogenitic properties, of the blood flowing into the side-branches. Thus, the protector device shown in FIG. 8, and therein generally designated 40, is provided with two end sections 41, 42 preferably having a relatively high porosity index, e.g., ranging between 65-95%, preferably, 75-95%; and an intermediate section 43 having a relatively low porosity index, e.g., ranging between 20 and 95%, preferably, 20-80%, to produce a diverting zone “F”. Thus, when the protector device 40 is implanted in the aortic arch, as shown in FIG. 5 or 7, the protector device is deployed such that its end zone 41 is anchored in the ascending aorta 21 covering the atheroma 23 therein, its opposite end zone 42 is anchored in the descending aorta 22, and its diverting zone 43 covers the orifices of the side-branches 25-27 to divert embolic material from entering into the side-branches, without substantially impeding the flow of the blood, or increasing the thrombogenitic properties of the blood, flowing into the side-branches.

A braided protector device as illustrated in FIG. 8 may be constructed by producing a pitch in the filaments in the intermediate diverting zone 43 which is smaller than the pitch l₁ in the end zones 41, 42. Thus, the porosity of the intermediate diverting zone 43 is larger than in the end zones 41, 42, to enable the diverting zone 43 to divert the ambolic material present in the blood from entering into the side-branches, while securely anchoring the protector device to the lumen at its opposite ends, and also securely covering the atheroma 23.

While the invention has been described with respect to several preferred embodiments, it will be appreciated that these are set forth merely for purposes of example, and that many other variations of the invention may be made. For example, instead of providing the protector device in the form of a braided tube, other mesh-like structures could be used, such as woven, knitted or cellular tubes. Also, the protector device could be composed of multiple tubular meshes lying one above the other in layer-like formations.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### BIBLIOGRAPHY

1. Davila-Roman V G, Barzilai B, Wareing T H, Murphy S F, Schechtman K B, Kouchoukos N T: Atherosclerosis of the ascending aorta. Prevalence and role as an independent predictor of cerebrovascular events in cardiac patients. Stroke, Oct. 25, 1994 (10):2010-6.

2. Davila-Roman V G, Murphy S F, Nickerson N J, et al: Atherosclerosis of the ascending aorta is an independent predictor of long-term neurologic events and mortality. J Am Coll Cardiol 33: 1308-16, 1999.

3. Katz E S, Tunick P A, Rusinek H, Ribakove G, Spencer F C, Kronzon I: Protruding aortic atheromas predict stroke in elderly patients undergoing cardiopulmonary bypass: experience with intraoperative transesophageal echocardiography. J Am Coll Cardiol, Jul. 20, 1992 (1):70-7.

4. Amarenco P. Duyckoerts C. Tzurio C: The frequency of ulcerated plaque in the aortic arch in patients with stroke. N Eng J Med 326:221-5, 1992.

5. French Study of Aortic Plaque in Stroke Group. Atherosclerotic disease of the aortic arch as a risk factor for recurrent ischemic stroke. N Eng J Med 334: 1216-21, 1996.

6. Stern A. Tunick P A, Calliford A T, et al: Protruding aortic arch atheroma, risk of stroke during heart surgery with and without aortic arch endarterectomy. Am Heart J. 128:746-52, 1999.

What is claimed is:

1. An implantable intraluminal device implantable in a body lumen having an atheroma therein in the vicinity of a side-branch orifice, comprising:
a mesh-like tube of bio-compatible material formed with liquid-permeable window openings;
said mesh-like tube having an expanded condition in which the tube diameter is slightly larger than the diameter of the body lumen in which it is to be implanted, and the tube length is sufficient to cover said atheroma and side-branch orifice to be anchored to the body lumen around the periphery of the atheroma;
said mesh-like tube also having a contracted condition wherein it is sufficiently flexible so as to be easily manipulatable through the body lumen to the site of the atheroma;
said mesh-like tube, in its expanded condition, having window openings of a size and distribution such as to:
structurally stabilize said atheroma and to keep embolic material originatable from the atheroma in place on the wall of said body lumen, while diverting embolic material of predetermined size present in the blood flowing through the mesh-like tube from said side-branch orifice, without substantially impeding the blood flow, or increasing the thrombogenitic properties, of the blood flowing into said side-branch orifice.

2. The implantable device according to claim 1, wherein said mesh-like tube has a porosity index in the expanded condition of the mesh-like tube within the range of 65-95%.

3. The implantable device according to claim 2, wherein said porosity index in the expanded condition of the mesh-like tube is within the range of 80-90%.

4. The implantable device according to claim 1, wherein said mesh-like tube has two end sections having a porosity index in in the expanded condition of the mesh-like tube within the range of 20-95% and a diverting section in between having a porosity index in the expanded condition of the mesh-like tube within the range of 65-95%.

5. The implantable device according to claim 1, wherein said mesh-like tube has two end sections having a porosity index in in the expanded condition of the mesh-like tube within the range of 20-80% and a diverting section in between having a porosity index in the expanded condition of the mesh-like tube within the range of 80-90%.

6. The implantable device according to claim 1, wherein said window openings in the expanded condition of the mesh-like tube are of a length along one side within the range of 100-1500 μm.

7. The implantable device according to claim 1, wherein said window openings in the expanded condition of the mesh-like tube are of a length along one side within the range of 400-1000 μm.

8. The implantable device according to claim 1, wherein said window openings in the expanded condition of the mesh-like tube have an inscribed diameter within the range of 30-800 μm.

9. The implantable device according to claim 1, wherein said window openings in the expanded condition of the mesh-like tube have an inscribed diameter within the range of 50-320 μm.

10. The implantable device according to claim 1, wherein said mesh-like tube is composed of a braid of filaments.

11. The implantable device according to claim 10, wherein the number of filaments in said braid is within the range of 60-300.

12. The implantable device according to claim 10, wherein the number of filaments in said braid is within the range of 100-160.

13. The implantable device according to claim 10, wherein said filaments have a circular cross-section of a diameter within the range of 20-500 μm.

14. The implantable device according to claim 10, wherein said filaments have a circular cross-section of a diameter within the range of 50-100 μm.

15. The implantable device according to claim 10, wherein said filaments have a non-circular cross-section of a circumference within the range of 60-800 μm.

16. The implantable device according to claim 10, wherein said mesh-like tube has, in its expanded condition, a porosity index within the range of 20-95%, window openings of a length along one side within the range of 100-1500 μm, a number of filaments within the range of 60-300, and a circular cross-section within the range of 20-500 μm.

17. The implantable device according to claim 10, wherein said mesh-like tube has, in its expanded condition, a porosity index within the range of 80-90%, window openings of a length along one side within the range of 400-1000 μm, a number of filaments within the range of 100-160, and a circular cross-section within the range of 50-100 μm.

18. The implantable device according to claim 1, wherein said device is made of a material selected from the group consisting of 316L stainless steel tantalum, superelastic Nitinol, cobalt base alloy, mixtures of such metals and alloys, bio compatible plastic and complex materials.

19. The implantable device according to claim 1, wherein said body lumen is the aortic arch.

20. The implantable device according to claim 2, wherein said mesh-like tube is of a length in its expanded condition such that its ends can be implanted in one or more of the body lumens of the ascending aorta, aortic arch, and or descending aorta.

21. The implantable device according to claim 1, wherein the mesh-like tube, in its expanded condition, exerts an average radial force of above 150 pascals on the wall of said body lumen.

22. A method for stabilizing atheromas in the aortic arch and preventing embolic material from being detached from the wall of said aortic arch, thus preventing physiological damage caused by protruding aortic arch atheroma, and also preventing emboli from proximal sources from entering into side-branches of said aortic arch, without substantially impeding the flow of blood through said aortic arch and said side-branches, comprising: implanting in said aortic arch an intraluminal device according to claim 1.

23. A method for stabilizing an atheroma in a body lumen and preventing embolic material from being detached from the atheroms, without substantially impeding the flow of blood through said lumen and a side-branch branching from said lumen, the method comprising: implanting an expandable mesh-like tube of a bio-compatible material in said body lumen to cover said atheroma; said mesh-like tube having a contracted state of a first diameter, and an expanded state of a second diameter greater than said first diameter, said mesh-like tube being flexible in its contracted state for manipulation through said body lumen to the implantation site and being suitable to keep said embolic material in place on said wall of said body lumen, said mesh-like tube being also suitable to divert embolic material passing therethrough from entering said side-branch.

24. The method according to claim 22, wherein said body lumen is the aortic arch.
